(19) United States
(12) Patent Application Publication
(10) Pub. No.: US 2024/0260950 A1
(43) Pub. Date: Aug. 8, 2024

(54) URINE COLLECTION CUP

(71) Applicant: FRIDABABY, LLC, Miami, FL (US)

(72) Inventors: Chelsea HIRSCHHORN, Miami, FL (US); Dana MCPHERSON, Miami, FL (US); Samuel SAUCEDA, El Serino, CA (US); Bailey DENTON, Miami, FL (US)

(73) Assignee: FRIDABABY, LLC, Miami, FL (US)

(21) Appl. No.: 18/106,050

(22) Filed: Feb. 6, 2023

(51) Int. Cl. A61B 10/00

(52) U.S. Cl. CPC A61B 10/007; A61B 10/0012; A61B 2560/0431

(57) ABSTRACT

A urine collection cup has a collapsible sidewall with a plurality of living hinges, such that the height of the cup in its collapsed configuration is much reduced. To reconfigure the collection cup into a use configuration, the user grasps a protruding bottom flange of the cup and pulls the flange downward and away from the cup's top rim. The cup may be part of a system that also includes a stick or strip for determining the presence or concentration of an analyte in the urine. An end of the stick may abut a ridge in the cup floor when the urine is being sampled. The top rim of the cup has opposed notches across which the stick or strip may be placed while undergoing a visual change.

### BACKGROUND OF THE INVENTION

At-home tests exist for women to tell if they are pregnant. Other tests are available to permit women to determine if they are ovulating, and therefore when they are most likely to conceive. Various body fluids can be sampled to determine this, and urine is one of these. A urine sample is applied to a stick or strip, and the stick or strip will undergo a visual change if a looked-for analyte is present, or if it is above a predetermined concentration.

Urine collection cups are known, and many are shaped in different ways to conform to the female anatomy. Known collection cups are also furnished with an elongate handle to aid in the positioning of the vessel to collect the urine.

It would be advantageous to provide a urine collection cup that occupies much less volume in a collapsed condition than it does in an expanded or use condition. It would further be advantageous to provide a urine collection cup with features for positioning a stick or strip to expose the stick or strip to the urine to be tested, and to then provide a rest while any visual change in the stick or strip becomes evident.

### SUMMARY OF THE INVENTION

According to one aspect of the invention, a urine collection cup is provided with a bottom wall and an endless sidewall. The sidewall upwardly extends from a junction with the bottom wall and is disposed around a vertical axis. The junction is radially inwardly displaced from a peripheral edge of the bottom wall, thereby creating an outer flange of the bottom wall.

The sidewall is configurable by the user into a collapsed configuration and into an expanded configuration suitable for use. The sidewall includes at least first, second and third sidewall segments. The first sidewall segment upwardly extends from the junction to a first living hinge. The second sidewall segment upwardly extends from the first living hinge to a second living hinge when the sidewall is in the expanded configuration. The third sidewall segment upwardly extends from the second living hinge. When the sidewall is in a collapsed configuration, the second segment downwardly extends from the first living hinge to the second living hinge. When the user wishes to reconfigure the sidewall from the collapsed configuration to the expanded configuration, the user grasps the outer flange of the bottom wall and pulls downward.

In one embodiment, the sidewall further includes a fourth sidewall segment which, when the sidewall is in the expanded configuration, upwardly extends from the third living hinge to a fourth living hinge. A fifth sidewall segment upwardly extends from the fourth living hinge. When the sidewall is in the collapsed configuration, the fourth sidewall segment downwardly extends from the third living hinge to the fourth living hinge.

In one embodiment, the sidewall is comprised of a folding portion and a frame or nonfolding portion. The nonfolding portion upwardly extends from a top margin of the folding portion. The first, second and third sidewall segments, and the first and second living hinges, are parts of the folding portion.

In one embodiment, an elongate handle is hingedly connected to the nonfolding portion of the sidewall. The handle is configurable to a use position in which the handle outwardly extends from the sidewall relative to the axis, and to a storage position in which the free end of the handle is disposed radially inwardly from the sidewall. In an embodiment, and when the handle is outwardly rotated from the storage position to the use position, a stop of the handle abuts the nonfolding portion of the sidewall to thereby provide a limit to the outward rotation of the handle and permit the handle to support the sidewall and bottom wall in space.

In an embodiment, a handle groove is formed in the sidewall to downwardly extend from a top rim thereof to a groove bottom. An outward rotation stop surface is disposed in the handle groove and extends from a left side of the handle groove to a right side of the handle groove. In one embodiment, the outward rotation stop surface is an outwardly facing surface of a stop wall. The stop wall has a top surface that is disposed below the top rim of the sidewall. The handle has a second stop. The abutment of the second stop of the handle with the top surface of the stop wall prevents further inward rotation of the handle beyond the storage position.

According to another aspect of the invention, a system for determining the presence or concentration of an analyte in a body fluid includes a cup and a stick. The cup is adapted to collect the body fluid, such as urine, and has a bottom wall. A sidewall upwardly extends from the bottom wall to a top rim and extends around a vertical axis. A first notch is formed in the top rim to have a first notch width. A second notch is formed in the top rim to have a second notch width and to be angularly spaced around the axis from the first notch. The first notch is also spaced from the second notch by a notch distance. An upstanding ridge is formed in the bottom wall to be radially inwardly spaced from the sidewall. A lower end of the stick is adapted to be placed in fluid collected in the cup and to abut the ridge. A length of the stick is greater than the notch distance, while a width of the stick is less than the first and second notch widths. The first and second notches are thereby adapted to receive the stick when the stick is placed on the top rim of the cup.

In one embodiment, the analyte is human chorionic gonadotropin (HCG) and the stick undergoes a visual change when it detects the presence of HCG in the urine. In another embodiment, the analyte is luteinizing hormone (LH) and the stick undergoes a visual change when the concentration of LH exceeds a predetermined amount.

The present invention thus provides an easily expandible and collapsible cup for receiving a body fluid. The cup has structural features optimizing it for use with a rod or stick for detecting an analyte.

### BRIEF DESCRIPTION OF THE DRAWINGS

Further aspects of the invention and their advantages can be discerned in the following detailed description as read in conjunction with the drawings of exemplary embodiments, in which like characters denote like parts and in which:

FIG. 1 is atop left perspective view of a urine cup according to the invention, shown in an expanded configuration and with a handle extended to a use position;

FIG. 2 is a rear side perspective view of the cup shown in FIG. 1:

FIG. 3 is a sectional view taken substantially along Line 3-3 of FIG. 1;

FIG. 4 is a sectional view taken substantially along Line 4-4 of FIG. 1;

FIG. 5 is a top left perspective view of the cup shown in FIG. 1, but in which a sidewall thereof is in a collapsed configuration and in which the handle is in a storage position;

FIG. 6 is top left perspective view similar to that shown in FIG. 5, but with the handle shown in a use position for the purpose of showing more detail;

FIG. 7 is a cross-sectional view taken substantially along Line 7-7 of FIG. 6;

FIG. 8 is a longitudinal sectional view taken substantially along Line 8-8 of FIG. 5;

FIG. 9 is a side sectional view of an upper or frame portion of a cup sidewall, with other parts removed to show detail;

FIG. 10 is across sectional view of the upper portion shown in FIG. 9;

FIG. 11 is a top right perspective view of the cup shown in FIG. 1, with the handle removed;

FIG. 12 is a top left perspective view of the cup shown in FIG. 1, as being used with a first test stick;

FIG. 13 is a top left perspective view of the cup and stick shown in FIG. 12, but with the stick being held by notches in the top rim;

FIG. 14 is a top left perspective view similar to FIG. 12, but showing the cup being used with a second test stick; and

FIG. 15 is a view similar to that shown in FIG. 13, but showing the cup supporting the second test stick.

### DETAILED DESCRIPTION

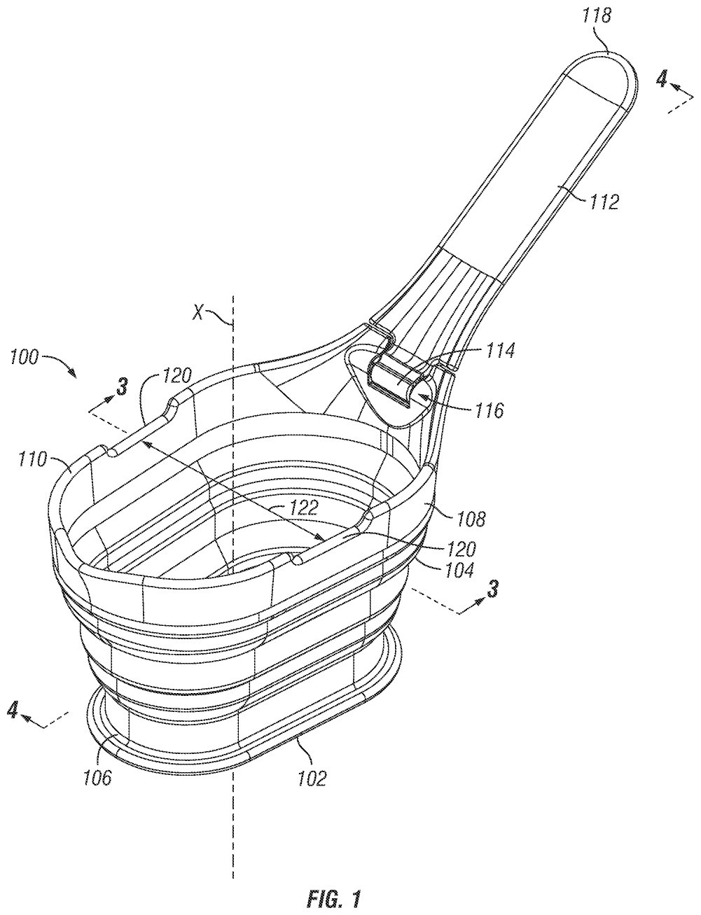

A cup for the collection of a body fluid such as urine is indicated generally by 100 in FIG. 1. Cup 100 has a bottom wall 102 and a sidewall 104 that upwardly extends from a junction 106 with the bottom wall 102. The sidewall 104 extends around a vertical axis X. An upper or frame portion 108 of the sidewall has a top rim 110, most of which conforms to a horizontal plane orthogonal to axis X.

In the illustrated embodiment, the rim 110, and the endless sidewall 104 more generally, are not circular but are oval or oblong in any horizontal plane. This is to better fit the female anatomy when it is desired to collect a urine sample. A handle 112 has a hinged end 114 attached to upper portion 108 at a hinge 116, the structure of which will be described in more detail below. Handle 112 is elongate and its length can be somewhat less than the largest horizontal dimension of the sidewall 104. FIG. 1 illustrates the hinged handle 112 is a use position, in which the free end 118 is radially more remote from axis X than is hinged end 114, and also is higher. This aids the user in positioning the cup 100.

Each long side of rim 110 had a notch or slot 120 in it. The notches 120 are angularly spaced from each other around axis X by 180 degrees, and are linearly separated from each other by a notch distance 122. Distance 122 is taken on a line that passes through axis X. A departure of rim 110 from the horizontal plane occurs at near hinge 116, where rim 110 rises to aesthetically blend into the handle 112 when the handle 112 is in the use position shown. Upper portion 108 may be slightly pointed at location 124, which can be opposite hinge 116. Location 124 therefore operates as a pour spout when it comes time to remove the urine being tested from cup 100.

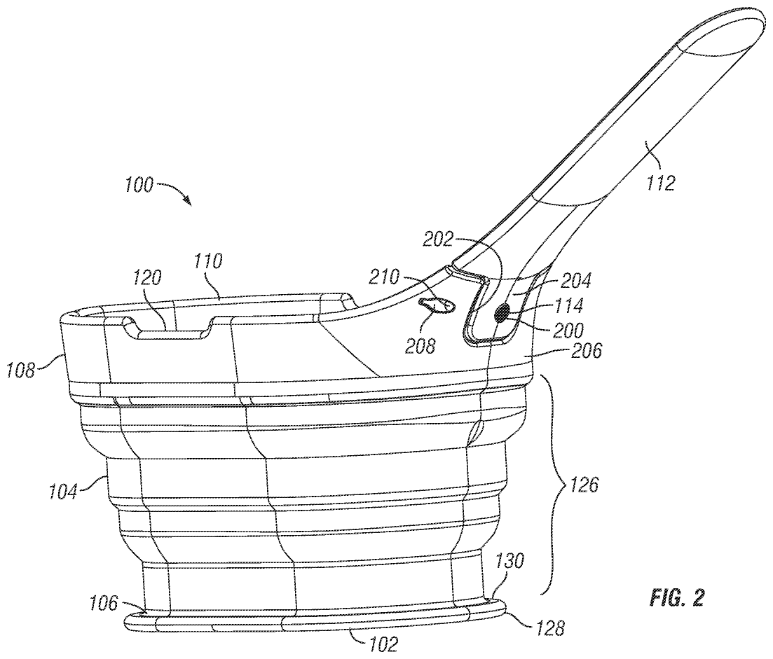

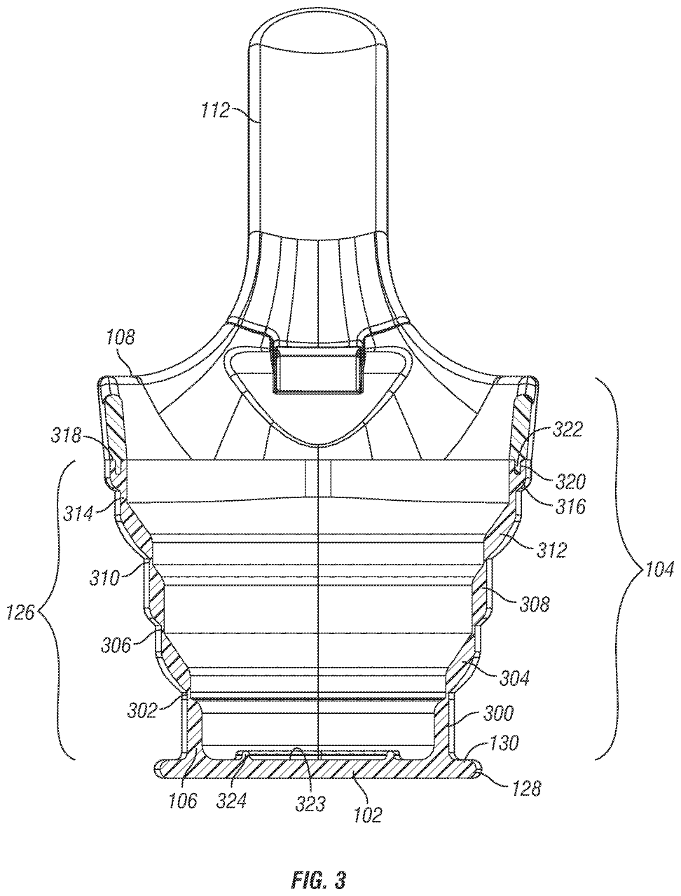

Further details of cup 100 are visible in FIG. 2. A lower portion 126 of the sidewall 104 has a plurality of sidewall segments in it, connected to each other by intervening living hinges, both of which will be described in more detail below. The bottom wall/sidewall junction 106 is radially inwardly spaced from a peripheral edge 128 of the bottom wall 102, thereby defining a peripheral flange 130.

Hinged end 114 of handle 112 takes the form of a tongue 200 that, in the use position, occupied a handle groove 202 formed in frame 108. An exterior surface 204 of the tongue 200 conforms to a general exterior surface 206 of the frame 108 when the handle 112 is in the use position. The handle groove 202 downwardly extends from top rim 110. A horizontal bore 208 is made in frame 108 to receive an axle or pin 210, which also is inserted through the hinged end 114 of the handle 112.

As seen in FIG. 3, the lower portion 126 of the cup sidewall may be integrally molded with the bottom wall 102 from an elastomer such as TPE or silicone rubber. The upper portion or frame 108 may be molded of a different polymer such as polypropylene. Lower portion 126 is flexible while frame or upper portion is relatively rigid. The sidewall lower portion 126 is constituted by a number of circumferential sidewall segments, joined together by horizontally disposed circumferential living hinges. A first sidewall segment 300 upwardly extends from junction 106 to a first living hinge 302. In the expanded configuration shown in FIG. 3, a second wall segment 304 upwardly and somewhat outwardly extends from first living hinge 302 to a second living hinge 306. A third sidewall segment 308 upwardly extends from second living hinge 306 to a third living hinge 310. In the expanded configuration shown in FIG. 3, a fourth sidewall segment 312 upwardly and somewhat outwardly extends from third living hinge 310 to a fourth living hinge 314. Finally, a fifth sidewall segment 316 upwardly extends from the fourth living hinge to a folding portion top margin 318.

Each of the living hinges 302, 306, 310, 314 is much thinner than the sidewall segments adjacent to it. For example, the thickness of living hinges 302, 306, 310 and 314 can be about 0.4 mm, while the nominal wall thickness of the sidewall segments 300, 304, 308, 312 and 316 may be about 2 mm. This causes the folding sidewall portion 126 to preferentially fold at the living hinges and not elsewhere. The folding portion top margin 318 has a downwardly extending groove 320 formed therein. This receives a downwardly extending tongue or blade 322 of the frame 108. In one embodiment, the folding portion 126 is overmolded on blade 322 of nonfolding portion 108.

An upper surface 323 of the bottom wall 102 is flat, with the exception of an upstanding ridge 324. The ridge 324 is radially inwardly displaced from the lower sidewall portion 126 and from sidewall segment 300 in particular. The ridge 324 may be endless and it may be disposed at a constant distance from sidewall segment 300, such as one in the range of 5 to 6 mm.

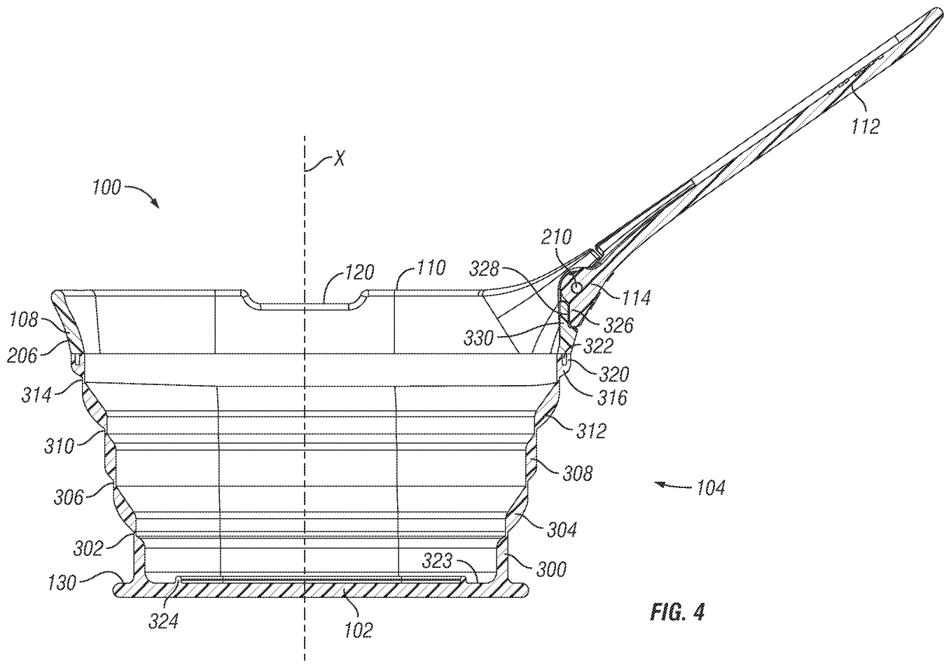

As seen in FIG. 4, hinged end 114 of handle 112 has a stop surface 326. When the handle 112 is in the use position as shown in this FIGURE, the stop surface 326 faces radially inwardly and is located below pin or axle 210. In the illustrated position, the stop surface 326 abuts an outwardly facing surface 328 of a stop wall 330. The stop wall 330 is molded as a part of the frame 108. The interaction of the stop surface 326 and stop wall surface 328 acts as a limit to the outward (in this view, clockwise) rotation of the handle 112 around pin 210. When placed in the illustrated use position, the handle 112, when grasped by the user, will be able to suspend the rest of cup 100 in space, including sidewall 104 and bottom wall 102. In the illustrated embodiment, the stop wall surface 328 is inwardly recessed from the general exterior surface 206 of frame 108, so that the lower/outer surface of handle end 114 will be substantially continuous with surface 206.

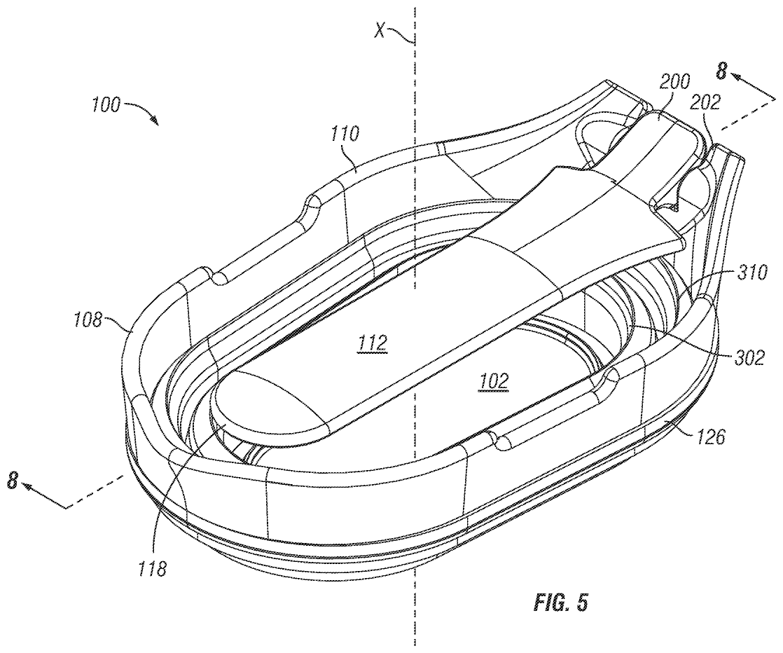

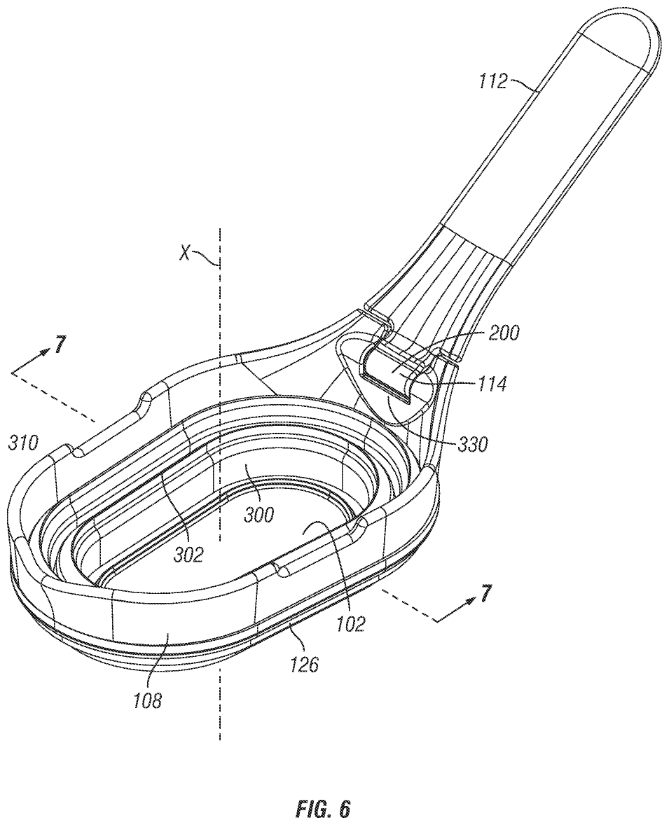

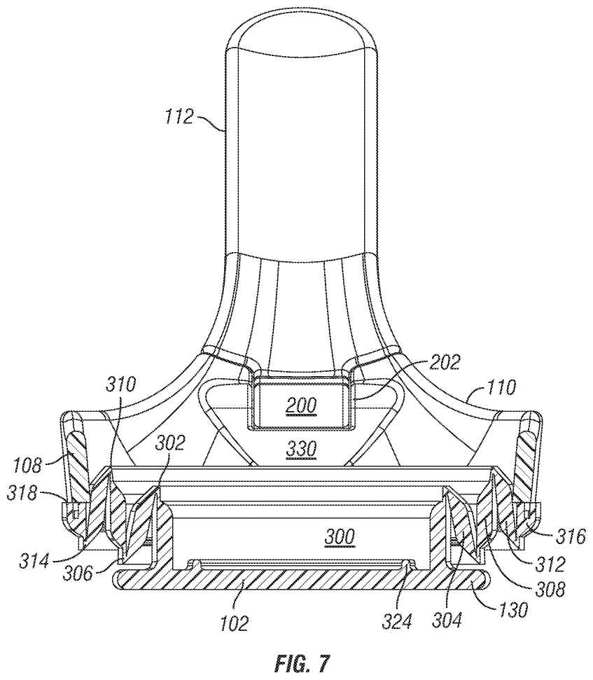

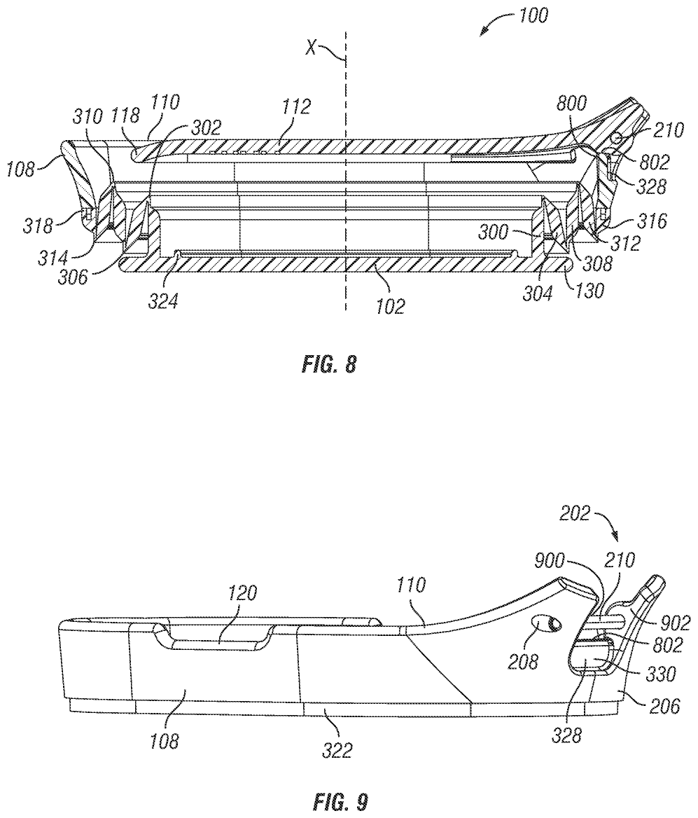

FIGS. 5 and 8 show cup 100 in a collapsed configuration, with handle 112 inwardly folded to a storage position. The sidewall segment 304 now extends downwardly and outwardly from living hinge 302 to living hinge 306. Similarly, the sidewall segment 312 now extends downwardly and outwardly from living hinge 310 to living hinge 314. This greatly diminishes the overall cup height. For maximum collapsibility, the sidewall segments 300, 304, 308 and 312 can all be of about the same vertical width.

Living hinge 302 is relatively close to axis X, while living hinges 306, 310 and 314 are progressively farther away. This allows the sidewall segments 304, 308, 312 and 316 to downwardly collapse without interfering with each other.

In the illustrated storage position, a downwardly facing stop surface 800 of handle 112 abuts a top surface 802 of the stop wall 328. The top surface 802 is disposed radially inwardly of pin 210. Therefore, the abutment of these two surfaces suspends handle 112 over the interior of cup 100. In the storage position, the handle 112 is at or below the cup top rim 110, and the free end 118 is closer to axis X than is any part of the frame 108.

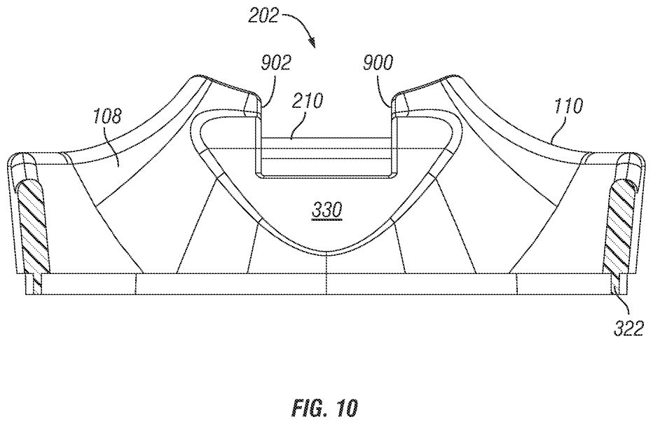

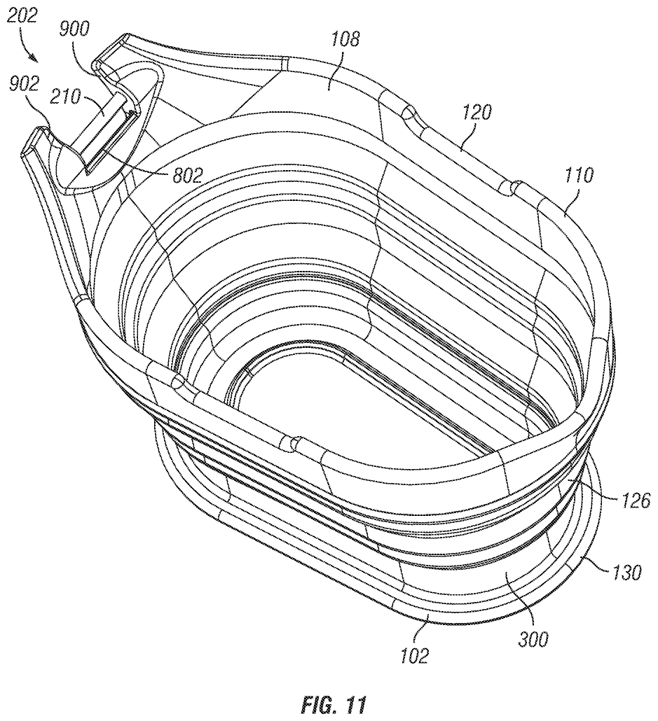

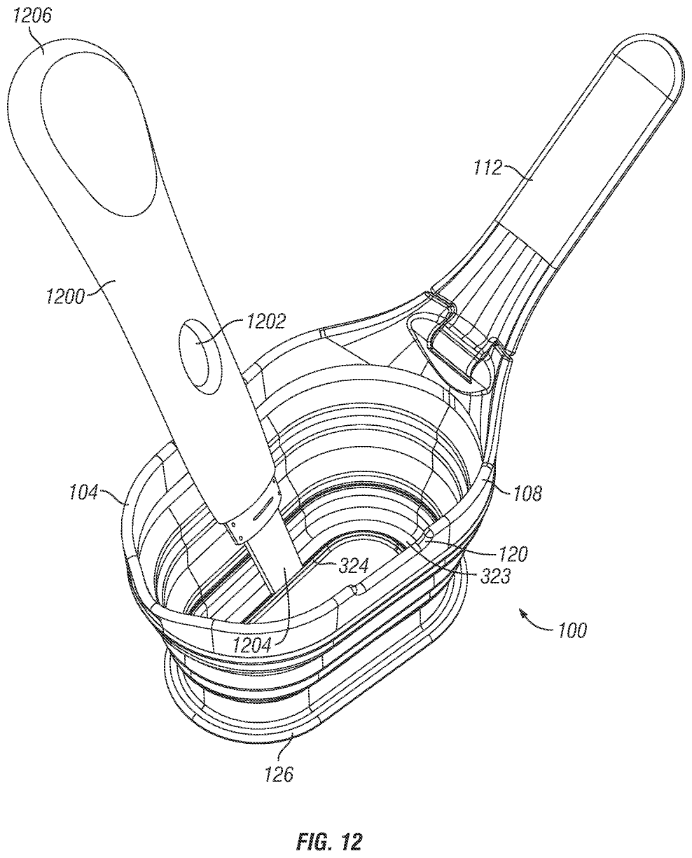

As seen in FIGS. 9-11, the stop wall 330 is substantially vertical and extends from a first side 900 of the handle groove 202 to a second side 902 of the handle groove 202. The exterior surface 328 of the stop wall 330 is inwardly recessed from the general exterior surface 206 of the frame 108.

The cup 100 may be provided to the user in the configuration shown in FIGS. 5 and 8, with the foldable sidewall portion 126 collapsed and the handle 112 rotated inwardly to its storage position. To use cup 100, the user grasps frame 108 and downwardly pulls on peripheral flange 130 until the sidewall segments 304, 312 rotate radially inwardly and then downwardly, assuming the positions seen in FIGS. 1-4. Before or after this, the user rotates the handle 112 from the storage position shown in FIGS. 5 and 8 to the use position shown in FIGS. 1-4. The cup 100 is then ready for use.

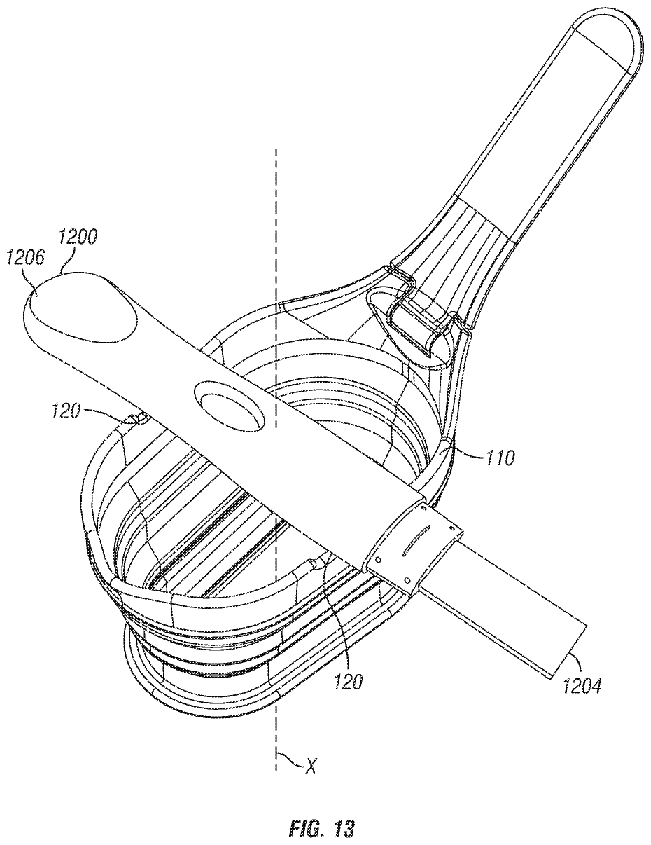

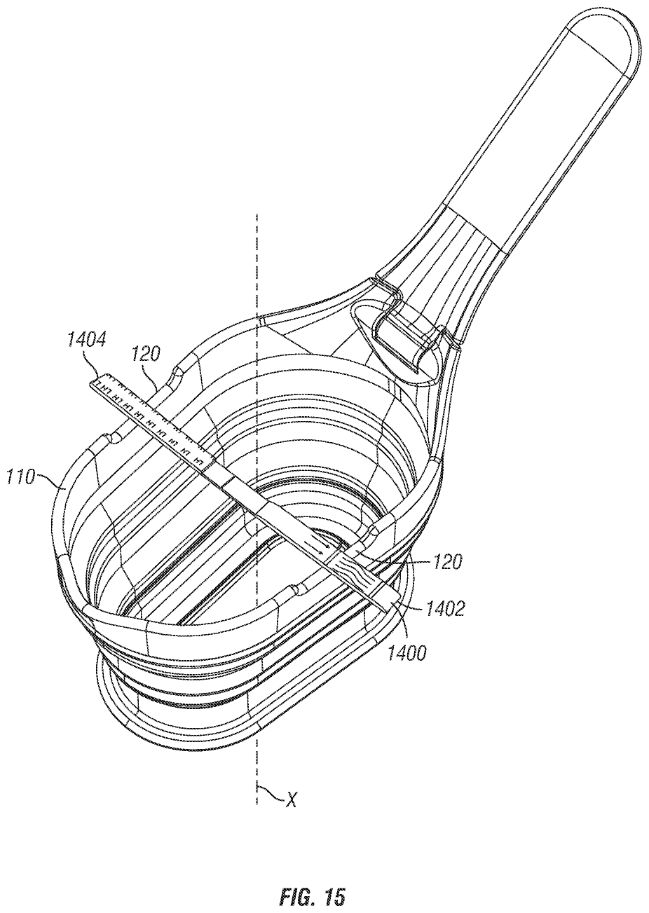

After collecting a urine sample, a test stick, such as stick 1200 or stick 1400 seen in FIGS. 12-15, is used to determine the presence or concentration of an analyte in the urine. These sticks may be of various sizes and may operate differently. Stick 1200 is a pregnancy test, is relatively large and has a window 1202 which changes color once it senses human gonadotropin hormone (HGH) in the urine. Stick or strip 1400, on the other hand, is meant to detect the surge in luteinizing hormone (LH) that occurs when a woman is ovulating. It therefore may be of the type that will not change visual appearance until a specific concentration of LH is met, or may indicate a relative concentration of this hormone by assuming any of several visual appearances. Other sticks or strips may be provided to detect the presence or concentration of other analytes.

To test the collected urine, an immersion end 1204, 1402 is placed in proximity with bottom wall upper surface 323 in the area between ridge 324 and sidewall 104, such that end 1204, 1402 abuts ridge 324. In this position, urine will contact the immersion end 1204, 1402. In the illustrated embodiment, ridge 324 is endless and follows the shape of the sidewall 104, but it could take other shapes and may have one or more ends. In any case, a length of the stick 1200, 1400 between the immersion end 1204, 1402 and a nonimmersion end 1206, 1404 should be greater than the height of the cup sidewall 104 when the cup sidewall folding portion 126 is in the expanded configuration. This permits the user to easily grasp the nonimmersion end 1206, 1404 of the stick 1200, 1400.

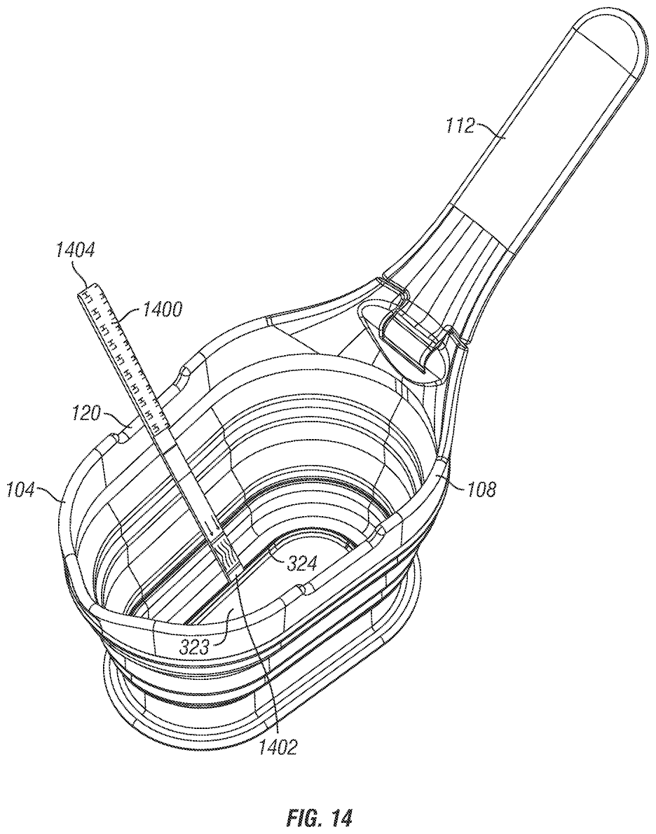

The user then places stick 1200 or stick 1400 across top rim 110 of cup 100, in the opposed notches 120 in rim 110. This is shown in FIGS. 13 and 15. In each case, the stick length is greater than notch distance 122 (FIG. 1), so that the stick may rest in both notches at the same time, spanning the cup interior. Further, the stick 1200, 1400 should have a width, orthogonal to its length, which is smaller than the width of rim notches 120. The stick 1200, 1400 is kept in this position until sufficient time has elapsed that the stick 1200 will show the presence of HGH, or until sufficient time has elapsed that the stick 1400 will determine that LH is present in the urine at or above a predetermined concentration.

In summary, a urine collection cup has been shown and described in which a user may grasp a bottom wall peripheral flange to pull a folding portion of the sidewall from a collapsed condition to an expanded condition. The cup further has features that optimize its use with test sticks for determining the presence or concentration of an analyte in the urine.

While illustrated embodiments of the present invention have been described and illustrated in the appended drawings, the present invention is not limited thereto but only by the scope and spirit of the appended claims.

We claim:

1. A urine collection cup comprising:
a bottom wall radially outwardly extending from a vertical axis to a peripheral edge;
an endless sidewall upwardly extending from a junction with the bottom wall and extending around the axis, the junction radially inwardly displaced from the peripheral edge to thereby define an outer flange of the bottom wall radially outwardly extending from the junction to the peripheral edge;
the sidewall configurable by a user into a collapsed configuration and into an expanded configuration;
the sidewall including first, second and third sidewall segments, the first sidewall segment upwardly extending from the junction to a first living hinge, the second sidewall segment upwardly extending from the first living hinge when the sidewall is in the expanded configuration to a second living hinge, the third sidewall segment upwardly extending from the second living hinge; wherein
the second sidewall segment, when the sidewall is in the collapsed configuration, downwardly extends from the first living hinge to the second living hinge, and wherein
the outer flange is adapted to be grasped by the user to downwardly pull the bottom wall and reconfigure the sidewall from the collapsed configuration to the expanded configuration.

2. The urine collection cup of claim 1, wherein the third sidewall segment upwardly extends to a third living hinge, the cup further comprising:
a fourth sidewall segment upwardly extending from the third living hinge when the sidewall is in the expanded configuration to a fourth living hinge, and a fifth sidewall segment upwardly extending from the fourth living hinge; wherein
the fourth sidewall segment, when the sidewall is in the collapsed configuration, downwardly extends from the third living hinge to the fourth living hinge.

3. The urine collection cup of claim 1, wherein the endless sidewall comprises a folding portion upwardly extending from the junction to a folding portion top margin, and a nonfolding portion upwardly extending from the folding portion top margin to a top rim of the sidewall, the first, second and third sidewall segments and the first and second living hinges being portions of the folding portion.

4. The urine collection cup of claim 3, wherein the folding portion and the nonfolding portion are molded of different polymeric materials, the nonfolding portion having a bottom margin joined to the top margin of the folding portion, one of the top margin of the folding portion and the bottom margin of the nonfolding portion having a groove, the other of the top margin of the folding portion and the bottom margin of the nonfolding portion having a tongue which fits within the groove.

5. The urine collection cup of claim 3, wherein the folding portion is molded of a thermoplastic elastomer.

6. The urine collection cup of claim 3, wherein an elongate handle is hingedly connected to the nonfolding portion, the handle extending from a hinged end disposed at the nonfolding portion of the sidewall to a free end, the handle configurable to a use position in which the handle outwardly extends from the sidewall relative to the axis, the handle further configurable to a storage position in which the free end of the handle is disposed radially inwardly from the sidewall.

7. The urine collection cup of claim 6, wherein the handle outwardly rotates from the storage position to the use position, a stop of the handle abutting the nonfolding portion of the sidewall in the use position to thereby provide a limit to the outward rotation of the handle and permit the support in space by the handle of the sidewall and of the bottom wall.

8. The urine collection cup of claim 1, wherein the sidewall upwardly extends to a top rim, the urine collection cup further comprising an elongate handle extending from a hinged end to a free end, the hinged end hingedly connected to the endless sidewall near the top rim, the handle configurable to a use position in which the handle outwardly extends away from the axis and to a storage position in which the free end of the handle is disposed radially inwardly from the top rim.

9. The urine collection cup of claim 8, wherein when the handle is in the use configuration, the handle extends upwardly from the top rim of the sidewall.

10. The urine collection cup of claim 8, wherein the handle outwardly rotates from the storage position to the use position, the sidewall having a substantially vertical outward rotation stop surface, a stop of the handle abutting the outward rotation stop surface of the sidewall when the handle is in the use position, thereby preventing further outward rotation of the handle and permitting the handle to suspend the sidewall and the bottom wall in space.

11. The urine collection cup of claim 10, wherein a handle groove is formed in the sidewall to downwardly extend from the sidewall top rim, the handle groove having a groove bottom downwardly displaced from the sidewall top rim, opposed left and right handle groove sides downwardly extending from the top rim of the sidewall to the groove bottom, the outward rotation stop surface extending from the left handle groove side to the right handle groove side.

12. The urine collection cup of claim 11, wherein the outward rotation stop surface is an outwardly facing surface of a stop wall extending from the left handle groove side to the right handle groove side, the stop wall having a top surface disposed below the top rim of the sidewall, a second stop of the handle abutting the top surface of the stop wall when the handle is in the storage position, the abutment of the second stop of the handle with the top surface of the stop wall preventing further inward rotation of the handle beyond the storage position.

13. A system for determining the presence or concentration of an analyte in a body fluid, the system comprising:
a cup having a bottom wall, a sidewall of the cup upwardly extending from the bottom wall and disposed to extend around a vertical axis, the sidewall upwardly terminating in a top rim, a first notch formed in the top rim and having a first notch width, a second notch formed in the top rim to be angularly spaced around the axis from the first notch and having a second notch width, the first notch linearly spaced from the second notch by a notch distance;
an upstanding ridge disposed in the bottom wall to be radially inwardly spaced from the sidewall; and
an elongate stick having an upper end and a lower end, the lower end adapted to be placed in fluid collected in the cup such that the lower end abuts the ridge, the stick having a stick length between the upper end and the lower end and a stick width orthogonal to the stick length;
the stick length being greater than the notch distance, the stick width being less than the first notch width and the second notch width, such that a user may place the stick on the top rim so as to be received in the first and second notches.

14. The system of claim 13, wherein the body fluid is urine and the analyte is luteinizing hormone, the stick undergoing a visual change when the concentration of the analyte in the urine exceeds a predetermined concentration.

15. The system of claim 13, wherein the bodily fluid is urine and the analyte is human chorionic gonadotropin, the stick undergoing a visual change when the analyte is detected.

16. The system of claim 13, wherein the sidewall of the cup is configurable to either an expanded configuration or a collapsed configuration, the sidewall having a height in the expanded configuration between the bottom wall and the top rim, the stick length being greater than the height of the sidewall.
